(19) United States
(12) Patent Application Publication
(10) Pub. No.: US 2010/0023125 A1
(43) Pub. Date: Jan. 28, 2010

(54) UNIVERSAL IMPLANT FOR THYROPLASTY

(75) Inventors: Christian Debry, Paris (FR); André Walder, L'Hay Les Roses (FR); Philippe Schultz, Oberschaeffolsheim (FR)

Correspondence Address: BUCHANAN, INGERSOLL & ROONEY PC, POST OFFICE BOX 1404, ALEXANDRIA, VA, 22313-1404, US

(73) Assignees: PROTIP SAS, Strasbourg (FR); UNIVERSITE LOUIS PASTEUR (ULP), Strasbourg (FR); HÔPITAUX UNIVERSITAIRES DE STRASBOURG (HUS), Strasbourg (FR)

(21) Appl. No.: 12/529,216

(22) PCT Filed: Feb. 29, 2008
(86) PCT No.: PCT/FR2008/000262

§ 371 Date: Aug. 31, 2009

(30) Foreign Application Priority Data
Mar. 1, 2007 (FR) 07/01482

(51) Int. Cl. A61F 2/20

(52) U.S. Cl. 623/14.11

(57) ABSTRACT

An implant being urged to be positioned between the thyroid cartilage (22) and a vocal cord (20) so as to displace the latter in order to restore the speech to a patient, characterized in that it comprises:
i) a “prosthesis” (3) being urged to rest on the muscle of the vocal cord (20),
ii) a “counterplate” (5) resting on the internal wall of the thyroid cartilage (22), and
iii) a liking element (7) of the prosthesis (3) and the counterplate (5),
   said counterplate (5) being mobile on said at least one linking element (7), thus enabling the adjustment of the distance D between the prosthesis (3) and the counterplate (5) to maintain the implant (1) in place.

The present invention concerns the domain of surgical implants and, more particularly, the domain of the implants being urged to correct a total or partial paralysis of the vocal cords so as to restore the speech to a patient. In a preferred manner, the invention is directed at a universal and adjustable implant used in thyroplasty.

Among the vocal paralysis, it can be mentioned the unilateral paralysis having mainly phonatory consequences and the bilateral paralysis resulting in, as for them, breathing problems. In both cases, some complications at the swallowing level occur.

The present invention is preferentially directed at, but without being limited to, the unilateral paralysis.

In the practice, such paralyses are generally due to laryngeal traumatisms, cancerous ailments or, more rare, infectious attacks.

The main consequences are the dysphonia, that is to say a blown voice, low or not voiced, due to an only partial compensation by the vocal cord not damaged. It is then necessary to overcome these dysfunctions by limiting the glottal leak and especially by increasing the tension of the damaged vocal cord.

It exists, to do so, various methods aiming at overcoming such a dysfunction of the vocal cords. It can be mentioned, as an example, endoscopic techniques such as the injection of teflon, of collagen, of macroplastic or of fat. If such injections are quick to implement and reproducible at will, they do not give complete satisfaction because it follows usually a vocal deterioration over time. Moreover, the difficulties linked to the injection result very often in a bad distribution of the product which is at the origin of an agglutination at the injection point.

More recently, it was developed several surgical techniques that have all as a common principle the insertion of an implant via an incision, also called window, made within the thyroid cartilage. It can be mentioned, as a matter of examples of such implants, the implants described by Isshiki Nobuhiko and, more recently, the implant described by Montgomery.

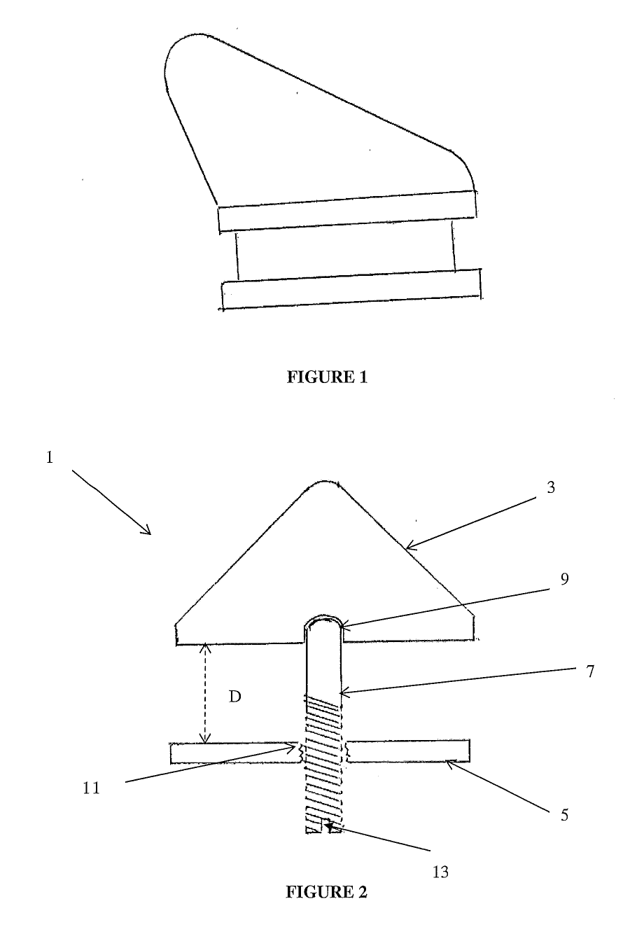

Concerning the latter, considered here as the closest prior art, it is about an implant consisting of a base in layers enabling its fixation in a cut having the shape of a window carried out in the thyroid cartilage and of a projecting element, having the shape of a triangle or “shark fin”, coming to press on the muscle of the vocal cord. This implant is described in the patent application published under the reference WO 92/21303. As it stands out from the description of this patent application, one of the characteristics of the implant called “of Montgomery” resides in the shape itself of the fixation base which comprises several layers, a lower layer, an intermediate layer and an upper layer, the lower layer and the upper layer having a bigger surface than the intermediate layer so as to facilitate the anchoring of the base in the window carried out within the thyroid cartilage. An example of this implant is represented on FIG. 1 and will be discussed further.

It is therefore necessary to open an aperture of a large size within the thyroid cartilage so as to enable the passage of such an implant. Moreover, the patient suffers an important traumatism at the level of this cartilage because of the fact that the latter can not close completely, the intermediate layer going right through it. Moreover, from such an anchorage, it exist discomfort risks, being able to go until the tearing of the cartilage, in case of displacement or of too high pressure.

At last, that implant being made of silicone and having a unique size, it is necessary to use a measuring instrument that consists of a projecting element having a similar form to that of the implant and a stopping element to determine the optimal size of the implant to put implement. Once the size is determined, it is then advisable to remove the measuring device and to implement the implant presenting the closest dimensions to the realized measurements.

The necessary resort to a measurement stage, not only increases the manipulations made by the practitioner as well as, consequently, the risks and troubles linked to the operation, but also the choice of an implant among a limited range does not enable to ensure the selection of an implant corresponding exactly to the space to be filled in between the thyroid cartilage and the vocal cord to be repaired. Lastly, it is necessary to control regularly to check that the size of the implant implemented is always the most appropriate.

The present invention has as an objective to overcome these disadvantages in proposing a universal implant, adjustable, capable of adapting itself to all the configurations and dimensions, more particularly to the distance between the thyroid cartilage and the muscle of the vocal cord.

More particularly, according to a first aspect, the invention has as an object a universal implant being urged to be positioned between the thyroid cartilage and a vocal cord so as to displace the latter in order to restore the speech to a patient, said implant being characterized in that it comprises:
i) a first element forming <<prosthesis>> being urged to rest on the muscle of the vocal cord resulting in a reaction force R of the vocal cord on said prosthesis,
ii) a second element forming “counterplate” being urged to rest on the internal wall of the thyroid cartilage resulting in a reaction force R′ of the thyroid cartilage on said counterplate, and
iii) at least a third linking element of the prosthesis and the counterplate,
   said counter-plate being mobile on said at least one linking element, thus enabling the adjustment of the distance D between the prosthesis and the counter-plate so as to be able to adjust the reaction forces R and R′ respectively exerted by the muscle of the vocal cord and by the thyroid cartilage to maintain the implant in place.

The implant object of the invention is revolutionary in that it enables for the first time, contrary to the whole prejudices of the man skilled in the art, to avoid that a portion of the implant goes through the thyroid cartilage. Indeed, the implant object of the invention is maintained in place only by the action of the reaction forces R and R′ exerted respectively by the muscle of the vocal cord and the thyroid cartilage.

More particularly, said reaction force R is exerted substantially perpendicularly to the plane AA′ defined by the longitudinal axis of the vocal cord while said reaction force R′ is exerted substantially perpendicularly to the plane BB′ defined by the surface of the internal wall of the thyroid cartilage.

That stability of the implant according to the invention is due to its capacity to be adjustable.

In the practice, the fact of being able to adjust in an very precise manner the distance D between the prosthesis and the counterplate enables to modulate in a very precise manner the reaction forces R and R′ so as they are sufficient to maintain the implant in place without however resulting in a discomfort for the patient nor a degradation of the thyroid cartilage or the muscle of the vocal cord.

Moreover, another advantage of the implant according to the invention is that the latter can be adjusted in the course of the time in order to adapt itself to any new deformation of the muscle of the vocal cord.

With the implants used to date, it was necessary to practice a heavy operation in order to remove the implant in place to enable its replacement by a implant of bigger size in case of a new distension of the muscle of the vocal cord or of release of the thyroid cartilage or by an implant of a smaller size in case of hardening of the muscle of the vocal cord or of the thyroid cartilage. Such an operation, it will be well understood, represents a disagreement and additional risks for the patient.

These important disadvantages do not exist anymore with the implant object of the invention. Indeed, for adjustment purposes it is not necessary anymore to practise such an operation of replacement of the implant, this one being able to simply be adjusted by an appropriate action at the level of the linking element so as to increase the distance between the prosthesis and the counterplate, named distance D, and thus to correctly adjust the implant in order to overcome any distension of the muscle of the vocal cord or of the thyroid cartilage.

It is obviously possible, in case of hardening of the muscle of the vocal cord as an example, to make the reverse operation by decreasing the distance D between the prosthesis and the counterplate.

It has to be understood that the invention rests on the principle of the indirect displacement of the prosthesis. In a more precise manner, the counterplate which is mobile on the linking element is urged to be positioned resting on the thyroid cartilage. When the surgeon acts on the linking element in order to increase the distance D, the counterplate is going to move towards the thyroid cartilage. The latter being rigid, it is going to oppose to the displacement of the counterplate, this will result in the increase of the reaction force R′ of the cartilage on said counterplate with as a consequence a displacement in the opposite way of the whole implant due to the fact of the resistance of the thyroid cartilage. It follows an increase of the reaction force R of the vocal cord on the implant object of the invention. The final result being that the implant is solidly maintained jammed between the thyroid cartilage and the vocal cord.

By acting in an opposite manner on the linking element, it ends up also at, in an indirect manner, to a decrease of the reaction force R exerted by the vocal cord.

According to a given embodiment of the invention, the implant according to the invention is characterized in that each of the three elements consist of distinct pieces.

Such an embodiment has as an advantage to enable if the case arises to replace only a part of the implant. Besides, it facilitates the implementation of the implant during the surgical operation.

However, it is agreed that any realization in one or two distinct pieces must also be understood as being an integral part of the invention.

A preferred embodiment of the invention will be described afterwards.

More particularly, the implant according to the invention is characterized in that said at least one linking element consists of a rod comprising a threaded portion, said threaded portion being urged to cooperate with the counterplate forming nut so as to be able to displace the latter and to adjust the distance D with the prosthesis.

The fact of using only one linking element appear as evident to a man skilled in the art, in the sense that it enables to limit the elements foreign to the human body. This enables also to decrease the cost of production of the implant. However, the resort to several linking elements, although not recommended, remains covered by the present patent application.

In this embodiment, it must be understood that the rod forming linking element presents at least one threaded portion acting as a screw in order to cooperate with a threaded opening provided for that purpose within the counterplate. It results in that said counterplate is mobile in translation along the linking element when it is rotating, in the same manner as a nut along a screw.

According to a more preferred embodiment, it is provided at the level of the section of the threaded end at least one element of cooperation with a tool intended to be able to act on the linking element. More particularly, this element of cooperation consists in a male or female print in particular a notch enabling the coupling to a tool, as for example a screwdriver, enabling to impart a rotation move to the rod forming linking element. By a rotating action on the linking element, it becomes then possible to displace the counterplate in translation. In the practice, and contrary to the implants of the prior art, it will be sufficient to practice a very small opening within the thyroid cartilage to enable a screwdriver to get through and thus to adjust the distance D.

Obviously, any equivalent device enabling an adjustment of the length of the linking element remains covered by the present invention.

According to another embodiment, it can be envisaged, as an example, to use a device comprising systems having shape memory such as springs being able to be compressed during the installation of the implant, those latter loosening once the implant is in place in order to generate reaction forces R and R′ to ensure the maintenance of the implant in place. Systems comprising magnets or a rack could also be envisaged.

Another characteristic of the invention is that, during the rotation movement of the rod forming linking element, it is necessary that the counterplate as the prosthesis are not, neither one nor the other are, drove by this movement and begin then to turn. This would have as an effect to damage the thyroid cartilage but mainly the muscle of the vocal cord.

Regarding the counterplate, during the rotation of the rod forming linking element, the reaction force R′ exerted by the thyroid cartilage increases. It results in an increase of the friction forces, this has as an effect to hamper any rotation of the counterplate on itself around the rod forming linking element.

Regarding the prosthesis, it is provided a mean of cooperation with the rod forming linking element enabling to avoid any driving of the rotating prosthesis.

More particularly, the implant according to the invention is characterized in that said threaded rod forming linking element comprises, at the level of the opposite end to the threaded end cooperating with the counterplate, a smooth portion being urged to be lodged in a housing intended for this purpose within the prosthesis so as to be able to turn freely without resulting in the rotation of the prosthesis.

The fact that the rod forming linking element presents a smooth surface and comes to lodge itself in freely within a housing also smooth on its internal surface enables, not to make those two elements interdependent; those latter being unable to cooperate within the framework of the rotation of one or the other. Of course it is only an illustration of one embodiment, the non cooperation in rotation of the rod forming linking element and of the prosthesis being able to be obtained by several other manners in particular by the use of a bearing.

On another side, this smooth surface of the same end of the linking element can extend beyond the part inserted in the housing of the prosthesis, creating an interruption of the threading and an increase of the section of the linking element which prevents that the linking element may accidently be taken out of the counterplate by making an unscrewing operation.

In the practice, according to a preferred embodiment, the dimensions of the housing within the prosthesis are slightly bigger than those of the section of the rod forming linking element so as to avoid any friction force between those two elements. It follows that the rod forming linking element will turn freely within said housing without driving with it the prosthesis.

The linking element, if it does not result in a rotation of the prosthesis further to its own rotation will however be displaced by translation and will then come to push said prosthesis resulting also in a translation movement of the latter.

According to an additional embodiment, it can be envisaged to be able to decrease easily the distance D.

To do so, said housing can consist of a maintenance device of the linking element once the latter is inserted.

Any device enabling to make interdependent the rod forming linking element with the prosthesis in order to, during the withdrawal of the rod forming linking element the latter does not go out of the housing but drives the prosthesis in its translation must be considered as being an integral part of the invention.

A preferred embodiment consists of the said maintenance device being a shoulder intended for within the housing, said shoulder cooperating with at least one element projecting at the level of the smooth end of the linking element.

Another embodiment can consist in providing a system of hooks or simply to use magnet pieces.

According to another aspect of the invention, the implant according to the invention is characterized in that said prosthesis is made of porous titanium, preferentially microporous.

The use of microporous titanium has the advantage of enabling a colonization of the prosthesis by the surrounding cells while limiting the rejects of the implant by the organism. It constitutes a rigid support for the growing and is therefore destined to stay more or less definitely in the organism. This point is of a great importance because it enables to overcome a major disadvantage of the prosthesis made of silicone used to date. Indeed, the silicone deteriorated quickly and not enabling an appropriate colonization of the implant, it is necessary to control its state regularly and to change the implant if the case arises, with the disadvantages and risks that this implies for the patient.

In a general manner, such a material consists of a three-dimensional juxtaposition of not alloyed titanium beads (preferentially as the titanium T40 or any alloy of titanium biocompatible) of which the diameter is generally comprised between 200 and 650 micrometers. The cavities between the beads are interconnected making an open porosity generally of the order of 30 to 40%. It can be mentioned, as an illustration of such a material, the French patent application n° 2 758 974 or the European patent EP 0 856 299 B1. Of course, the above-mentioned elements are given as illustrative examples and any equivalent material known by the man skilled in the art can be used according to the invention in particular any biocompatible material such as the titanium foams, the solid titanium, the coral, the silicone, the bone powder.

The realization of the prosthesis in microporous titanium can be available in several embodiments. It can be in particular envisaged, without being compulsory, to drown in the basis of the prosthesis a plate made of solid titanium pierced with a smooth hole forming the housing in which the smooth portion of the threaded rod is urged to lodge. In a more economical embodiment the housing is made directly in the prosthesis made of porous titanium without addition of any other piece.

The present invention is also directed at, in a general manner, the use of an implant according to the invention for the treatment of dysfunctions of the vocal cords.

More particularly, such dysfunctions consist of a total or partial paralysis of the vocal cords.

At last, according to a last aspect, the present invention claims also a process that is simple and not much restrictive for the implementation of such an implant.

More particularly, the present invention concerns at last a process of implementation and/or of adjustment of an implant according to the invention. Said process consisting in placing said implant in order that the counterplate (5) is urged to form a support against the internal wall of the thyroid cartilage and
i) results in a displacement of the linking element (7) in order to increase the distance D increasing this way, according to the resistance offered by said thyroid cartilage, the reaction force R′, that has as an effect to displace said prosthesis resulting thus in an increase in the pressure exerted on the muscle of the vocal cord; or
ii) results in the displacement of said linking element in order to decrease the distance D decreasing this way the reaction force R′, that has as an effect, according to the reaction force R′ exerted by the muscle of the vocal cord on the whole constituted by said at least one linking element and by said prosthesis, to decrease the pressure exerted on the muscle of the vocal cord.

The invention will be better understood after the reading of the examples of realization described here-below in combination with the following figures in which:

The FIG. 1 illustrates the prior art and represents an implant of Montgomery in transverse section,

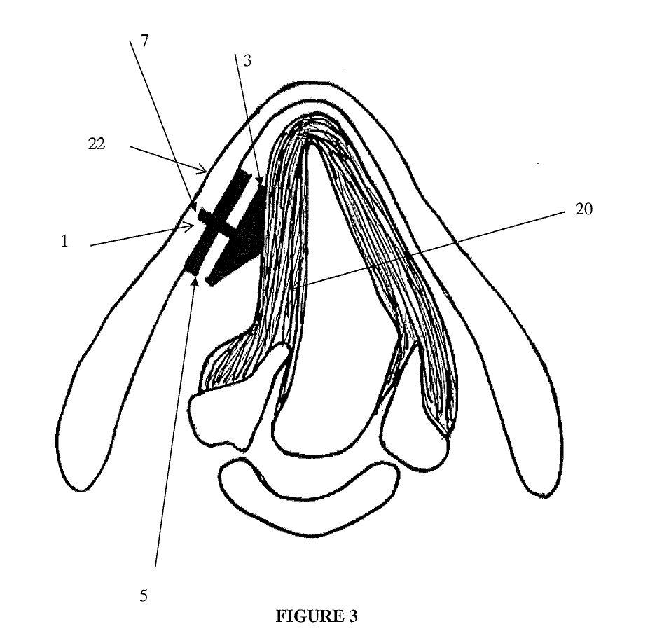

The FIG. 2 represents a preferred embodiment of an implant in transverse section according to the invention,

The FIG. 3 represents the implant according to the invention as implemented on a patient, and

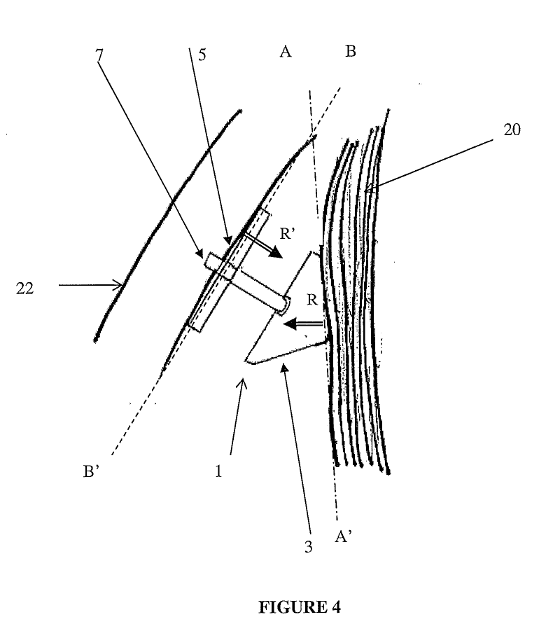

The FIG. 4 represents, in more details, the disposition of the implant according to the invention between the thyroid cartilage and the damaged vocal cord.

As described above, and as it stands out from FIG. 1, the implant described in the document of the closest prior art, namely the implant of Montgomery, comprises a portion slightly in the shape of a shark fin intended to come exerting a pressure on the damaged vocal cord and a second portion intended to maintain the whole implant in place within the thyroid cartilage. This second portion consists of three parts, a central part inserted through the thyroid cartilage and two other parts, respectively on each side of said central part, coming to rest on each of the sides of the thyroid cartilage in order to act as a stop and thus maintain the implant in place.

As it stands out from the FIG. 2, the implant 1 according to the invention presents a totally innovative form in the sense that it consists of a first element forming prosthesis 3, a second element forming counterplate 5 and a third element forming linking element 7. The linking element 7 consists of a threaded portion, in the manner of a screw, that is urged to be screwed within an opening 11 intended for within the counterplate 5, said opening 11 being also threaded on its internal walls, in the manner of a nut. At the level of the end of the threaded portion of the linking element 7, is also provided a notch 13. It is then possible to displace the element 7 through the counterplate 5 in the manner of a screw in a nut by simple action with a screwdriver being urged to be lodged in the notch 13 intended for that effect.

The opposite end of the linking element 7 is urged to be lodged within a housing 9 intended for that effect within the prosthesis 3. As it stands out from the FIG. 2, the linking element 7 turns freely within the housing 9. To do so, the dimensions of the housing 9 are slightly superior to those of the end of the linking element 7 in order to the latter is able to turn with the minimum friction on the internal walls of said housing 9.

The principle of the implant 1 according to the invention appears clearly in view of the FIG. 2, meaning that in acting on the linking element 7, it should have as an effect to result in the rotation of the counterplate 5 increasing thus the distance D. The counterplate 5 being maintained in place against the thyroid cartilage (not represented), it will not be able to move or enter in rotation, this will have as an effect to displace the linking element 7 in the reverse direction which will then come to push the prosthesis 3 resulting then in an increase of the pressure exerted by the prosthesis 3 on the muscle of the damaged vocal cord (not represented). In other words by screwing, or unscrewing according to the direction of the thread, the linking element (7) on the counterplate 5, the linking element 7 displaces the prosthesis 3 towards the muscle of the vocal cord that is itself displaced and brought closer to the second vocal cord. The use of a screw enables thus the adjustment with precision of the position of the muscle of the damaged vocal cord in order to enable it to recover its functionality.

The functioning of the prosthesis object of the present invention stands out more clearly from FIGS. 3 and 4 described hereafter.

According to the represented embodiment, the prosthesis 3 presents, in transverse section, a general profile having the shape of an isosceles triangle. More particularly, the dimensions intended for the basis of such an isosceles triangle can be comprised between 10 and 15 mm, and more preferentially in the example represented of 13 mm. The height, or median, of said triangle can be comprised between 4 and 6 mm. In a preferred manner, at least the summit of said triangle coming to rest on the muscle of the vocal cord is rounded so as to avoid any damaging of said vocal cord. It can be envisaged a curvature radius for this summit of the order of 3 mm. Of course, it has to be understood that the whole dimensions given above are only representing an illustrative example of an embodiment and that those dimensions are not to be taken as limitative.

The FIG. 3 is a general view, in horizontal section, of the implant 1 according to the invention mounted between the thyroid cartilage 22 and the muscle of the damaged vocal cord 20 in order to displace the latter.

It stands out clearly from this figure that only a small portion of the linking element 7 can remain inserted within the thyroid cartilage 22. It will then be sufficient for the surgeon to open a slight notch within said cartilage 22 to enable to slide a screwdriver and this way “stretch again”, or “release”, the implant 1 in order to adjust the distance D between the prosthesis 3 and the counterplate 5. This aspect presents a considerable advantage in comparison with the prior art where it was necessary to practice a big opening within the thyroid cartilage 22 in order to enable the withdrawal of the implant in place in its integrity to replace it by a new implant of the approximate appropriate size.

The FIG. 4 represents a magnification of the FIG. 3. More particularly, this figure shows the implant 1 positioned in a patient.

The counterplate 5 is resting on amongst the thyroid cartilage 22 according slightly to the plane BB′. The reaction force exerted by the thyroid cartilage 22 on the counterplate 5 is represented by the vector R′. On the opposite of the counterplate 5, the prosthesis 3 comes to rest on, as to it, on the muscle of the vocal cord to be stretched again 20 according slightly to the plane AA′. The latter exerts then a reaction force on the prosthesis 3, said force being represented by the vector R. It has to be noted that the representation of the reaction forces R and R′ in the form of vectors is only illustrative in order to understand the principle of the invention; the dimensions of these vectors R and R′ on the present FIG. 4 are thus not to be taken in consideration. It is then obvious for the man skilled in the art that by adjusting the distance D between the counterplate 5 and the prosthesis 3, it is possible to modify and modulate the reaction forces R and R′ in order to not only maintain the implant 1 in place but also to displace sufficiently the vocal cord 20 by pressure in order to restore, at least partly, the speech to the patient.

The embodiments of the invention described above consist of, of course, preferred embodiments. Any improvement not resulting in a substantial modification of the principle of the invention must be considered as covered by the present patent application and, particularly, by the claims hereafter that define its scope.

1. A universal implant (1) being urged to be positioned between the thyroid cartilage (22) and a vocal cord (20) so as to move the latter in order to restore the speech to a patient, the implant comprises:
i) a first element forming <<prosthesis>> (3) being urged to rest on the muscle of the vocal cord (20) resulting in a reaction force R of the vocal cord on said prosthesis,
ii) a second element forming counterplate (5) being urged to rest on the internal wall of the thyroid cartilage (22) resulting in a reaction force R′ of the thyroid cartilage on said counterplate, and
iii) at least one third linking element (7) of the prosthesis (3) and the counterplate (5),
said counterplate (5) being mobile on said at least one linking element (7), thus enabling the adjustment of the distance D between the prosthesis (3) and the counterplate (5) in order to be able to adjust the reaction forces R and R′ exerted respectively by the muscle of the vocal cord (20) and by the thyroid cartilage (22) to maintain the implant (1) in place.

2. The implant (1) according to claim 1, wherein each of the three elements (3, 5, 7) consist of distinct pieces.

3. The implant (1) according to claim 1, wherein at least one linking element (7) consists of a rod comprising a threaded portion, said threaded rod being urged to cooperate with the counterplate (5) forming nut in order to be able to displace the linking element (7) and to adjust the distance D comprised between the counterplate (5) and the prosthesis (3).

4. The implant (1) according to claim 1, wherein the threaded rod forming linking element (7) comprises, at the level of the opposite end to the threaded end cooperating with the counterplate (5), a smooth portion being urged to be lodged in a housing (9) envisaged for this purpose within the prosthesis (3) so as to be able to turn freely without resulting in the rotation of the prosthesis (3).

5. The implant (1) according to claim 1, wherein the prosthesis (3) is made of porous titanium, preferentially microporous.

6. The implant (1) according to claim 5, wherein the prosthesis (3) consists of a solid element pierced with a hole forming the housing (9).

7. The implant (1) according to claim 2, wherein at least one linking element (7) consists of a rod comprising a threaded portion, said threaded rod being urged to cooperate with the counterplate (5) forming nut in order to be able to displace the linking element (7) and to adjust the distance D comprised between the counterplate (5) and the prosthesis (3).

8. The implant (1) according to claim 2, wherein the threaded rod forming linking element (7) comprises, at the level of the opposite end to the threaded end cooperating with the counterplate (5), a smooth portion being urged to be lodged in a housing (9) envisaged for this purpose within the prosthesis (3) so as to be able to turn freely without resulting in the rotation of the prosthesis (3).

9. The implant (1) according to claim 3, wherein the threaded rod forming linking element (7) comprises, at the level of the opposite end to the threaded end cooperating with the counterplate (5), a smooth portion being urged to be lodged in a housing (9) envisaged for this purpose within the prosthesis (3) so as to be able to turn freely without resulting in the rotation of the prosthesis (3).

10. The implant (1) according to claim 2, wherein the prosthesis (3) is made of porous titanium, preferentially microporous.

11. The implant (1) according to claim 3, wherein the prosthesis (3) is made of porous titanium, preferentially microporous.

12. The implant (1) according to claim 4, wherein the prosthesis (3) is made of porous titanium, preferentially microporous.
